(19) United States
(12) Patent Application Publication
(10) Pub. No.: US 2015/0144802 A1
(43) Pub. Date: May 28, 2015

(54) WATER PURIFICATION AND WATER SUPPLY SYSTEM DECONTAMINATION APPARATUS

(71) Applicant: Corning Incorporated, Corning, NY (US)

(72) Inventor: Anthony Sebastian Bauco, Horseheads, NY (US)

(73) Assignee: Corning Incorporated, Corning, NY (US)

(21) Appl. No.: 14/160,711

(22) Filed: Jan. 22, 2014

Related U.S. Application Data
(60) Provisional application No. 61/908,246, filed on Nov. 25, 2013.

(51) Int. Cl. C02F 1/32; A61L 2/10; C02F 1/74

(52) U.S. Cl. CPC C02F 1/325; C02F 1/74; A61L 2/10; C02F 2201/3224; C02F 2305/10; C02F 2303/04; C02F 2307/14; C02F 2103/06
USPC 250/435; 250/492.1

(57) ABSTRACT

A water sanitizing system including a supply system having a supply conduit with a conduit length; a light-diffusing optical fiber in the conduit that substantially spans the conduit length; and an ultraviolet light source configured to inject ultraviolet light rays into the fiber. The fiber includes: (a) a first end and a second end, the ends defining a fiber length, (b) a core region comprising fused silica having a plurality of scattering sites, and (c) a cladding over the core region, the cladding having an outer photocatalyst region doped with a metal oxide. The cladding may comprise a polymer coating. The fiber is configured to propagate the light rays from the first end toward the second end of the fiber, and scatter the rays in substantially radial directions out of the core region of the fiber at the plurality of scattering sites, and through the photocatalyst region.

### CROSS-REFERENCE TO RELATED APPLICATION

This application claims the benefit under 35 USC §119(e) of provisional application Ser. No. 61/908,246, filed Nov. 25, 2013, entitled WATER PURIFICATION SUPPLY SYSTEM DECONTAMINATION APPARATUS, the entire contents of which are incorporated by reference.

### FIELD

The present disclosure generally relates to the field of water purification and decontamination and, more specifically, water sanitizing systems that employ light-diffusing fibers (“LDF”).

### BACKGROUND

Obtaining safe drinking water from personal wells, and from other sources, is a challenge in the United States and other countries throughout the world. Bacteria- and pesticide-related contamination often affects the quality and safety of these water sources. Typically, bacteria-related contamination is treated through the introduction of chemicals into the water sources. For example, chlorine and potassium sulfate are often added to wells to improve the quality and ensure the safety of water obtained from these wells. These chemicals can be toxic, costly and difficult to obtain in some countries.

Ultraviolet (“UV”) light can also be used to treat water sources subject to bacterial contamination. While UV light is effective at killing bacteria in a quantity of water, its effectiveness is limited to the small volume of the overall water source centered around the light source employed in the system. Another problem associated with conventional UV light-based sanitizing systems is that they cannot treat water sources with multiple contamination sources located in different parts of the water source system. In addition, conventional UV light-based systems do not address pesticide-related contamination that may have leeched into the water table associated with the water source.

Accordingly, there is a need for a less toxic, relatively low cost and effective water sanitizing system that can be used to treat bacterial- and pesticide-related contamination in water sources, particularly at various locations within systems containing and distributing water from these sources.

### SUMMARY

According to one embodiment, an optical fiber for sanitizing a water supply system is provided that includes a light-diffusing optical fiber. The fiber comprises: (a) a length, (b) a core region comprising fused silica having a plurality of scattering sites, and (c) a cladding over the core region, the cladding having an outer photocatalyst region doped with a metal oxide. The fiber is configured to propagate ultraviolet light rays along the length, and scatter the ultraviolet light rays in substantially radial directions out of the core region of the fiber at the plurality of scattering sites, through the photocatalyst region.

According to another embodiment, an optical fiber for sanitizing a water supply system is provided that includes a light-diffusing optical fiber. The fiber comprises: (a) a length, (b) a core region comprising fused silica having a plurality of scattering sites, and (c) a cladding over the core region that comprises a polymer coating. The fiber is configured to propagate ultraviolet light rays along the length, and scatter the ultraviolet light rays in substantially radial directions out of the core region of the fiber at the plurality of scattering sites.

According to a further embodiment, a water sanitizing system is provided. The water sanitizing system includes: a supply system having a water supply conduit with a conduit length; a light-diffusing optical fiber in the conduit that substantially spans the conduit length; and an ultraviolet light source configured to inject ultraviolet light rays into the optical fiber. The light-diffusing optical fiber includes: (a) a first end and a second end, the ends defining a fiber length, (b) a core region comprising fused silica having a plurality of scattering sites, and (c) a cladding over the core region, the cladding having an outer photocatalyst region doped with a metal oxide. The fiber is configured to propagate the ultraviolet light rays from the first end toward the second end of the fiber, and scatter the ultraviolet light rays in substantially radial directions out of the core region of the fiber at the plurality of scattering sites, and through the photocatalyst region.

According to an additional embodiment, a water sanitizing system is provided. The water sanitizing system includes: a water supply system having a water supply conduit with a conduit length; a light-diffusing optical fiber in the conduit that substantially spans the conduit length; and an ultraviolet light source configured to inject ultraviolet light rays into the optical fiber. The light-diffusing optical fiber includes: (a) a first end and a second end, the ends defining a fiber length, (b) a core region comprising fused silica having a plurality of scattering sites, and (c) a cladding over the core region that comprises a polymer coating. The fiber is configured to propagate the ultraviolet light rays from the first end toward the second end of the fiber along the fiber length, and scatter the ultraviolet light rays in substantially radial directions out of the core regions of the fiber at the plurality of scattering sites.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiments, and together with the description serve to explain principles and operation of the various embodiments.

### BRIEF DESCRIPTION OF THE DRAWINGS

FIG. 1 is a schematic cross-sectional view of a light-diffusing optical fiber having a cladding with an outer photocatalyst region according to an exemplary embodiment;

FIG. 1A is schematic perspective view of the fiber depicted in FIG. 1, configured with a UV light source and light delivery fiber according to another exemplary embodiment;

FIG. 2 is a schematic cross-sectional view of a light-diffusing optical fiber having a cladding with a polymer coating according to a further exemplary embodiment;

FIG. 2A is schematic perspective view of the fiber depicted in FIG. 2, configured with a UV light source and light delivery fiber according to an exemplary embodiment;

FIG. 3 is a schematic view of a water sanitizing system that utilizes one or more of the light-diffusing optical fibers depicted in FIGS. 1 and 2 according to a further exemplary embodiment;

FIG. 4 is a schematic view of a water sanitizing system employed in a residential plumbing system according to another exemplary embodiment; and

FIG. 5 is a schematic view of a water sanitizing system employed in a well system according to an additional exemplary embodiment.

### DETAILED DESCRIPTION

Reference will now be made in detail to the present preferred embodiments, examples of which are illustrated in the accompanying drawings. Whenever possible, the same reference numerals will be used throughout the drawings to refer to the same or like parts. It should be understood that the embodiments disclosed herein are merely examples, each incorporating certain benefits of the present disclosure.

Various modifications and alterations may be made to the following examples within the scope of the present disclosure, and aspects of different examples may be mixed in different ways to achieve yet further examples. Accordingly, the true scope of the disclosure is to be understood from the entirety off the present disclosure, in view of but not limited to the embodiments described herein.

Terms such as “horizontal,” “vertical,” “front,” “back,” etc., and the use of Cartesian Coordinates are for the sake of reference in the drawings and for ease of description and are not intended to be strictly limiting either in the description or in the claims as to an absolute orientation and/or direction.

In the description of the invention below, the following terms and phrases are used in connection to light-diffusing fibers.

The “refractive index profile” is the relationship between the refractive index or the relative refractive index and the waveguide (fiber) radius.

The “relative refractive index percent” is defined as:

Δ(r)%=100×[n(r)²−(n_{REF})²]/2n(r)²,

where n(r) is the refractive index at radius, r, unless otherwise specified. The relative refractive index percent Δ(r) % is defined at 850 nm unless otherwise specified. In one aspect, the reference index n_{REF} is silica glass with the refractive index of 1.452498 at 850 nm. In another aspect, n_{REF} is the maximum refractive index of the cladding glass at 850 nm. As used herein, the relative refractive index is represented by Δ and its values are given in units of “%”, unless otherwise specified. In cases where the refractive index of a region is less than the reference index n_{REF}, the relative index percent is negative and is referred to as having a depressed region or depressed-index, and the minimum relative refractive index is calculated at the point at which the relative index is most negative unless otherwise specified. In cases where the refractive index of a region is greater than the reference index n_{REF}, the relative index percent is positive and the region can be said to be raised or to have a positive index.

An “up-dopant” is herein considered to be a dopant which has a propensity to raise the refractive index of a region of a light-diffusing optical fiber relative to pure undoped SiO₂. A “down-dopant” is herein considered to be a dopant which has a propensity to lower the refractive index of a region of the fiber relative to pure undoped SiO₂. An up-dopant may be present in a region of a light-diffusing optical fiber having a negative relative refractive index when accompanied by one or more other dopants which are not up-dopants. Likewise, one or more other dopants which are not up-dopants may be present in a region of a light-diffusing optical fiber having a positive relative refractive index. A down-dopant may be present in a region of a light-diffusing optical fiber having a positive relative refractive index when accompanied by one or more other dopants which are not down-dopants.

Likewise, one or more other dopants which are not down-dopants may be present in a region of a light-diffusing optical fiber having a negative relative refractive-index.

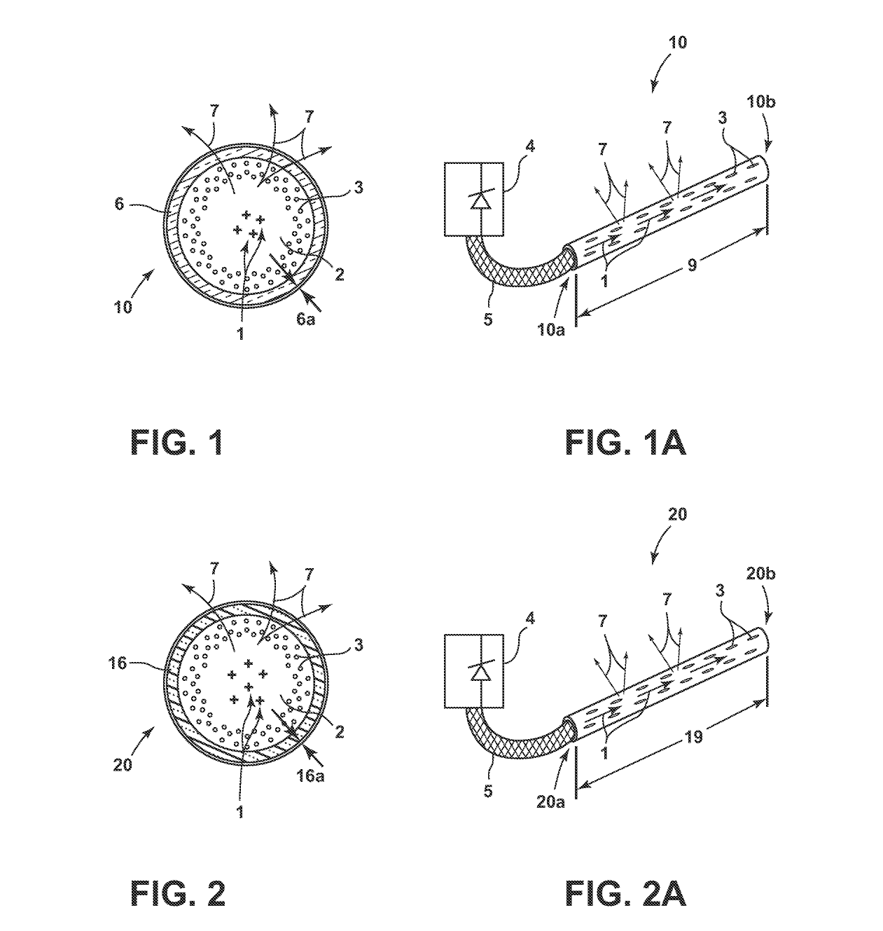

Referring to FIGS. 1 and 1A, a light-diffusing optical fiber 10 is depicted according to one exemplary embodiment. The fiber 10 is configured for sanitizing a water supply system and includes a first end 10a and a second end 10b. The ends 10a and 10b define a length 9. Light-diffusing optical fiber 10 further includes a core region 2 and a cladding 6 over the core region 2.

The core region 2 of the fiber 10 depicted in FIGS. 1 and 1A substantially comprises a fused silica glass composition with an index of refraction, n_{core}. In some embodiments, n_{core} is about 1.458. The core region 2 may have a radius ranging from about 20 μm to about 1500 μm. In some embodiments, the radius of the core region 2 is from about 30 μm to about 400 μm. In other embodiments, the radius of the core region 2 is from about 125 μm to about 300 μm. In still other embodiments, the radius of the core region 2 is from about 20 μm, 30 μm, 40 μm, 50 μm, 60 μm, 70 μm, 80 μm, 90 μm, 100 μm, 120 μm, 140 μm, 160 μm, 180 μm, 200 μm, 220 μm, 240 μm, 260 μm, 280 μm, 300 μm, 400 μm, 500 μm, 600 μm, 700 μm, 800 μm, 900 μm, 1000 μm, 1100 μm, 1200 μm, 1300 μm, 1400 μm or 1500 μm.

Still referring to FIGS. 1 and 1A, the core region 2 further includes a plurality of scattering sites 3. These scattering sites 3 are located in a scattering region within the core region 2 of light-diffusing optical fiber 10. These scattering sites 3 may comprise gas-filled voids or gaseous pockets (e.g. air-filled pockets), such as taught by U.S. application Ser. Nos. 12/950,045, 13/097,208, 13/269,055, and 13/713,224, herein incorporated by reference. In other embodiments, scattering sites 3 can comprise particles, such as micro- or nanoparticles of ceramic materials, configured to scatter UV light. It is preferable to select a medium for scattering sites 3 that demonstrates little absorption in the UV wavelengths (approximately 10 nm to 450 nm), for example, SiO₂ particles.

When gas-filled voids are employed for the plurality of scattering sites 3 in the core region 2, these voids may be distributed throughout the core region 2. The gas-filled voids employed as scattering sites 3 may also be located at the interface between core region 2 and the cladding 6, or they may be arranged in an annular ring within core region 2. The gas-filled voids may be arranged in a random or organized pattern and may run parallel to the length 9 of the fiber 10 or may be helical in shape (i.e., rotating along the long axis of the fiber 10 along the length 9). The scattering region within the core region 2 that contains the scattering sites 3 may comprise a large number of gas-filled voids, for example more than 50, more than 100, or more than 200 voids in the cross-section of the fiber 10. In other embodiments, the scattering sites 3 may comprise gas-filled voids at a volume fraction of about 0.1 to 30% in the core region 2. For embodiments of optical fiber 10 having a particularly long length, e.g., on the order of approximately 100 m, the volume fraction of gas-filled voids employed as scattering sites may approach zero to ensure sufficient propagation of light rays 1 down the length of the fiber without appreciable loss to the desired scattering locations. Further, in some embodiments, it is advantageous to vary the volume fraction of gas-filled voids as a function of fiber length to change the degree of light scattering at different locations of the fiber, depending on the application.

The gas-filled voids may contain, for example, SO₂, Kr, Ar, CO₂, N₂, O₂, or mixtures thereof. The cross-sectional size (e.g., approximate diameter) of the voids may be from about 1 nm to about 1 μm, or in some embodiments, the cross-sectional size may range from about 1 nm to about 10 μm. The length of each gas-filled void may vary from about 1 μm to about 100 m, in some cases dependent on the overall length 9 of the fiber 10. In some embodiments, the cross-sectional size of the voids employed as scattering sites 3 is about 1, nm, 2 nm, 3, nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 400 nm, 500 nm, 600 nm, 700 nm, 800 nm, 1 μm, 2 μm, 3 μm, 4 μm, 5 μm, 6 μm, 7 μm, 8 μm, 9 μm, or 10 μm. In other embodiments, the length of the voids is about 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm, 5 mm, 10 mm, 50 mm, 100 mm, 500 mm, 1 m, 5 m, 10 m, 20 m, 30 m, 40 m, 50 m, 60 m, 70 m, 80 m, 90 m, or 100 m.

The scattering sites 3 in the core region 2 of the light-diffusing optical fiber 10 are configured to scatter UV light rays 1 propagating within the core region 2 along the axial direction of the fiber 10. In particular, these sites 3 scatter the light rays 1 in substantially radial directions—i.e., as scattered UV light rays 7 outward from the core region 2, and through the cladding 6 and photocatalyst region 6a of the fiber 10. These scattered UV light rays 7 illuminate the light-diffusing optical fiber 10 in the UV spectrum in the space surrounding the fiber 10. In turn, these scattered UV light rays 7 can be employed to kill bacteria and other microbes in the water in proximity to the fiber 10, at least along the full length 9 of the fiber 10.

As also depicted in FIGS. 1 and 1A, a UV light source 4 can be connected to the first end 10a of the light-diffusing optical fiber 10 by a delivery fiber 5. Suitable light sources for UV light source 4 include conventional high-brightness LED sources. The delivery fiber 5 can be a single fiber, a bundle of fibers or a single large étendue fiber that is subsequently spliced or coupled to a bundle of light diffusing fibers 10. Preferably, the delivery fiber 5 is configured to propagate UV light rays 1 without significant scattering and absorption at the UV wavelengths. In other embodiments, the UV light source 4 is directly connected to the first end 10a of the fibers 10, thereby eliminating the need for a delivery fiber.

The scatter-induced attenuation associated with voids employed as scattering sites 3 in the core region 2 of the fiber 10 may be increased by increasing the concentration of the these voids, positioning the voids throughout the fiber 10, or in cases where the voids are limited to an annular ring-shaped region, by increasing the width of the annulus comprising the voids. In some embodiments in when the gas-filled voids employed as scattering sites 3 are helical in shape, the scattering-induced attenuation may also be increased by varying the pitch of the helical voids over the length of the fiber 10. Specifically, it has been found that helical voids with a smaller pitch scatter more light than helical voids with a larger pitch. Accordingly, the intensity of the illumination of the fiber 10 along its length 9 can be controlled (i.e., predetermined) by varying the pitch of the helical voids along the axial length 9. As used herein, the “pitch” of the helical voids refers to the inverse of the number of times the helical voids are wrapped or rotated around the long axis of the fiber 10 per unit length.

Referring again to FIGS. 1 and 1A, the light-diffusing optical fiber 10 further includes a cladding 6 arranged over the core region 2. The cladding 6 of fiber 10 further comprises an outer photocatalyst region 6a, located in proximity to the outer surface of the cladding 6. As such, cladding 6 is preferably comprised of silica glass. It also preferable to employ a glass composition for cladding 6 with a low refractive index to increase the numerical aperture (“NA”) of the fiber 10. In some embodiments, the cladding 6 may comprise silica glass down-doped with fluorine, boron or a combination of these dopants. The NA of the fiber 10 may be from about 0.12 to about 0.30 for some embodiments, and may range from about 0.2 to about 0.3 for other embodiments. In other embodiments, the relative refractive index of the cladding may be less than −0.5%, and in still others less than −1%.

In light-diffusing optical fibers 10, the cladding 6 generally extends from the outer radius of the core region 2. In some embodiments, the thickness of the cladding 6 is greater than about 5 μm, greater than about 10 μm, greater than about 15 μm or greater than about 20 μm. In other embodiments, the cladding 6 has a thickness of about 5 μm, 10 μm, 15 μm, 20 μm, 25 μm, or 30 μm. In still other embodiments, the thickness of cladding 6 ranges from about 5 μm to about 30 μm.

For light-diffusing optical fibers 10, the overall fiber diameter (i.e., the diameter of core region 2 plus the thickness of cladding 6) ranges from about 125 μm to about 3000 μm. In further embodiments, the optical fibers 10 have an overall diameter that ranges from about 45 μm to about 3000 μm. In other embodiments, the optical fibers 10 have an overall diameter of about 45 μm, 50 μm, 60 μm, 70 μm, 80 μm, 90 μm, 100 μm, 125 μm, 150 μm, 175 μm, 200 μm, 225 μm, 250 μm, 275 μm, 300 μm, 350 μm, 400 μm, 450 μm, 500 μm, 600 μm, 700 μm, 800 μm, 900 μm, 1000 μm, 1500 μm, 2000 μm, 2500 μm, or 3000 μm.

Referring again to FIGS. 1 and 1A, the cladding 6 of light-diffusing optical fiber 10 also includes an outer photocatalyst region 6a. The photocatalyst region 6a is doped with a metal oxide. Preferably, the metal oxide is selected such that it interacts with scattered UV light rays 7 to break down pesticides in proximity to the fiber 10. In some embodiments of light-diffusing optical fiber 10, outer photocatalyst region 6a can be doped with TiO₂ and/or ZnO. The total dopant concentration levels in the photocatalyst region 6a are preferably maintained in the range of about 1 to about 20 weight %. In addition, the thickness of photocatalyst region 6a may range from about 0.1 μm to about 10 μm. In some embodiments, the thickness of the photocatalyst region 6a is about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, or 10 μm.

Because light-diffusing optical fibers 10 operate in UV wavelengths and possess a photocatalyst region 6a, they can advantageously be utilized to kill bacteria and microbes in water in proximity to the fiber 10, while at the same time purifying the water by breaking down pesticides. As such, light-diffusing fibers 10 are particularly configured to propagate UV light rays 1 at UV wavelengths.

In some embodiments described herein, the light-diffusing optical fibers 10 will generally have a length 9 from about 100 m to about 0.15 m. In some embodiments, the fibers 10 will generally have a length 9 of about 100 m, 75 m, 50 m, 40 m, 30 m, 20 m, 10 m, 9 m, 8 m, 7 m, 6 m, 5 m, 4 m, 3 m, 2 m, 1 m, 0.75 m, 0.5 m, 0.25 m, 0.15 m, or 0.1 m. Generally, the fibers 10 are tailored with a length 9 based on the dimensions of the water source, conduits and/or plumbing hosting the fibers 10 for purposes of water sanitizing and purification.

Further, the light-diffusing optical fibers 10 described herein have a scattering-induced attenuation loss of greater than about 0.1 dB/m and up to about 20 dB/m at UV wavelengths, including at a wavelength of 300 nm, 325 nm, 350 nm, 375 nm, 400 nm, 425 nm, or 450 nm. For example, in some embodiments, the scattering-induced attenuation loss may be greater than about 0.1 dB/m, 0.2 dB/m, 0.3 dB/m, 0.4 dB/m, 0.5 dB/m, 0.6 dB/m, 0.7 dB/m, 0.8 dB/m, 0.9 dB/m, 1 dB/m, 1.2 dB/m, 1.4 dB/m, 1.6 dB/m, 1.8 dB/m, 2.0 dB/m, 2.5 dB/m, 3.0 dB/m, 3.5 dB/m, 4 dB/m, 5 dB/m, 6 dB/m, 7 dB/m, 8 dB/m, 9 dB/m, 10 dB/m, or 20 dB/m at UV wavelengths including at a wavelength of 300 nm, 325 nm, 350 nm, 375 nm, 400 nm, 425 nm, or 450 nm.

As described herein, the light-diffusing optical fibers 10, depicted in FIGS. 1 and 1A, may be constructed to produce uniform illumination of UV light (e.g., scattered UV light rays 7) along the entire length 9 of the fiber 10, or uniform illumination along a segment of the fiber 10 which is less than its entire length 9. The phrase “uniform illumination,” as used herein, means that the intensity of light emitted from the fiber 10 does not vary by more than 25% over the specified length. Uniform illumination of fibers 10 can be particularly important for some applications of fibers 10 to ensure that the UV light rays 7 are well-distributed throughout the host water source, and its components, to ensure effective purification and sanitizing of the water in the source.

Referring to FIGS. 2 and 2A, another exemplary embodiment of a light-diffusing optical fiber 20 is depicted. The optical fibers 20 are configured for sanitizing a water supply system. Light-diffusing optical fibers 20 are similar to the light-diffusing optical fibers 10 depicted in FIGS. 1 and 1A. Commonly identified elements associated with the fibers 20, such as the core region 2, are identical to those same elements employed in connection with the light-diffusing optical fibers 10. Unless otherwise noted, the properties and attributes of fibers 10 discussed earlier (e.g., scattering-induced attenuation loss, fiber length, distribution of scattering sites 3, etc.) apply equally to light-diffusing optical fibers 20.

Further, the light-diffusing optical fibers 20 include a first end 20a and a second end 20b. The ends 20a and 20b of fibers 20 define a length 19. In addition, a UV light source 4 can be connected to the first end 20a of the light-diffusing optical fiber 20 by a delivery fiber 5. Suitable light sources for UV light source 4 include conventional high-brightness LED sources. The delivery fiber 5 can be a single fiber, a bundle of fibers or a single large étendue fiber that is subsequently spliced or coupled to a bundle of light diffusing fibers 20.

The primary difference between light-diffusing optical fibers 10 and 20 is that fibers 20 lack an outer photocatalyst region (see, e.g., FIG. 1, photocatalyst region 6a) within their cladding 16. Instead, the fibers 20 depicted in FIGS. 2 and 2A have a cladding 16 over the core region 2 that comprises a polymer coating 16a. Because the light-diffusing optical fibers 20 do not possess a photocatalyst region, they cannot be used to remove pesticides from a water source through the interaction of UV light, a photocatalyst and the pesticide. However, the fibers 20 can be used for anti-microbial purposes. In addition, the polymer coating 16a employed with the cladding 16 makes the fibers 20 particularly suitable for movement and insertion in various geometries within components of a water supply system. In particular, the polymer coating 16a gives the fibers 20 added flexibility and better lubricity for insertion into various components of a water supply system, including small diameter pipes.

Referring again to FIGS. 2 and 2A, the light-diffusing optical fiber 20 further includes a cladding 16 arranged over the core region 2. Cladding 16 employed with the fibers 20 is generally comparable to the cladding 6 employed in light-diffusing optical fibers 10 (see FIGS. 1 and 1A). As shown in FIGS. 2 and 2A, the cladding 16 of fiber 20 further comprises a polymer coating 16a, located on the outer surface of the cladding 16. As such, cladding 16 is preferably comprised of silica glass. It also preferable to employ a glass composition for cladding 16 with a low refractive index to increase NA of the fiber 20. In some embodiments, the cladding 16 may comprise silica glass down-doped with fluorine, boron or a combination of these dopants. In other embodiments, cladding 16 may comprise a polymeric composition. In some cases, the polymeric composition employed for cladding 16 is comparable to that employed for polymer coating 16a. When the cladding 16 comprises a polymeric composition, the NA of the fiber 20 may be greater than about 0.3 and up to about 0.5 for some embodiments, and may range from about 0.39 to about 0.53 for other embodiments. In other embodiments of fiber 20 having a cladding 16 comprising a polymeric composition, the relative refractive index of the cladding may be less than −0.5%, and in still others less than −1%. Conversely, when the cladding 16 comprises a glass composition, the NA of the fiber 20 may be from about 0.12 to about 0.30 for some embodiments, and may range from about 0.2 to about 0.3 for other embodiments. In other embodiments, the relative refractive index of the cladding may be less than −0.5%, and in still others less than −1%.

In light-diffusing optical fibers 20, the cladding 16 generally extends from the outer radius of the core region 2. In some embodiments, the thickness of the cladding 16 is greater than about 5 μm, greater than about 10 μm, greater than about 15 μm or greater than about 20 μm. In other embodiments, the cladding 16 has a thickness of about 5 μm, 10 μm, 15 μm, 20 μm, 25 μm, or 30 μm. In still other embodiments, the thickness of cladding 16 ranges from about 5 μm to about 30 μm.

For light-diffusing optical fibers 20, the overall fiber diameter (i.e., the diameter of core region 2 plus the thickness of cladding 16) ranges from about 125 μm to about 3000 μm. In further embodiments, the optical fibers 20 have an overall diameter that ranges from about 45 μm to about 3000 μm. In other embodiments, the optical fibers 20 have an overall diameter of about 45 μm, 50 μm, 60 μm, 70 μm, 80 μm, 90 μm, 100 μm, 125 μm, 150 μm, 175 μm, 200 μm, 225 μm, 250 μm, 275 μm, 300 μm, 350 μm, 400 μm, 450 μm, 500 μm, 600 μm, 700 μm, 800 μm, 900 μm, 1000 μm, 1500 μm, 2000 μm, 2500 μm, or 3000 μm.

As also depicted in FIGS. 2 and 2A, the polymer coating 16a employed with the light-diffusing optical fibers 20 may comprise a clear layer of secondary coating comparable to the coatings typically employed in telecommunications fibers for mechanical handling. In some embodiments, polymer coating 16a is a layer coated on the outside surface of the cladding 16. In other embodiments, polymer coating 16a serves as the cladding 16 and is coated on the outside surface of core region 2. Such secondary coatings employed as polymer coating 16a are described in U.S. application Ser. No. 13/713,224, herein incorporated by reference. For polymer coating 16a employed in light-diffusing optical fibers 20, the thickness of the coating 16a can be minimized to reduce the amount of UV light absorption. In some embodiments, the polymer coating 16a can comprise an amorphous fluorinated polymer, such as Teflon® AF. In other embodiments, the polymer coating 16a can comprise an acrylate-based coating, such as CPC6, manufactured by DSM Desotech, Elgin, Ill. In some other embodiments, the polymer coating 16a can comprise a silicone-based polymer coating. In an additional set of embodiments, the polymer coating 16a can comprise a low refractive index polymeric material such as a UV- or thermally-curable fluoroacrylate, such as PC452 available from SSCP Co. Ltd., 403-2, Moknae, Ansan, Kyunggi, Korea.

In some embodiments of light-diffusing optical fibers 20, the thickness of the polymer coating 16a can range from about 1 μm to about 15 μm. In some embodiments, the thickness of the polymer coating 16a ranges from about 0.1 μm to about 50 μm, including thickness values of 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, or 50 μm. Preferably, the thickness of the polymer coating 16a is set at a range from about 5 μm to about 10 μm.

Light diffusing optical fibers 10 and 20 can be formed utilizing various techniques. For fiber embodiments in which the scattering sites 3 comprise gas-filled voids, these voids can be incorporated into the fibers by the methods described in U.S. application Ser. Nos. 11/583,098, 12/950,045, 13/097,208, 13/269,055, and 13/713,224, herein incorporated by reference. Generally, the light-diffusing optical fibers 10 and 20 are drawn from an optical fiber preform with a fiber take-up system and exit the draw furnace along a substantially vertical pathway (not shown). In some embodiments, fibers 10 and 20 are rotated as they are drawn to produce helical voids (serving as scattering sites 3) along the long axis 9, 19 of the fibers 10, 20, respectively. As the optical fibers 10 and 20 exit the draw furnace, a non-contact flaw detector may be used to examine the optical fiber for damage and/or flaws that may have occurred during the processing of the fibers. Thereafter, the diameter of the optical fibers 10 and 20 may be measured with a non-contact sensor. Optionally, the fibers 10 and 20 can be drawn through a cooling system which cools the optical fiber (not shown).

For light-diffusing optical fibers 20, the optional cooling step would be performed before the application of polymer coating 16a, and before the creation of cladding 16 when it comprises a polymeric composition. As the optical fibers 20 exit the cooling system, the fibers 20 enter at least one coating system where one or more polymer layers are applied to the cladding 16, thereby forming the polymer coating 16a. As the fibers 20 exit the polymer coating system, the diameter of the fibers can be measured using a non-contact sensor. Thereafter, a non-contact flaw detector can be used to examine the fibers 20 for damage and/or flaws in the cladding 16 and the polymer coating 16a that may have occurred during the manufacture of the fibers.

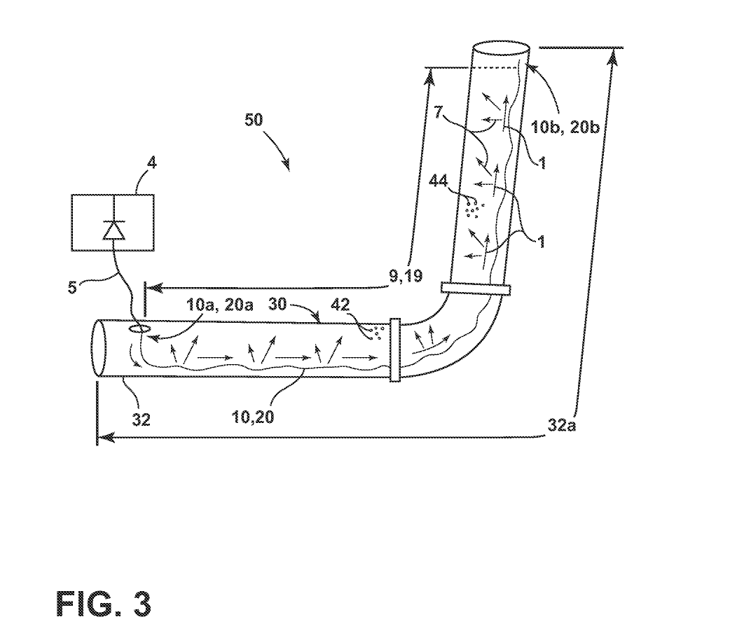

Referring to FIG. 3, light-diffusing optical fibers 10, 20 (see FIGS. 1, 1A, 2, 2A and corresponding description) can be employed in water sanitizing system 50 according to a further exemplary embodiment. In some embodiments, combinations of light-diffusing optical fibers 10 and/or 20 are employed in the sanitizing system 50 in loose bunches or tightly-wound bundles. The water sanitizing system 50 comprises a water supply system 30 that includes a water supply conduit 32 with a conduit length 32a. As shown in FIG. 3, the optical fibers 10, 20 substantially span the conduit length 32a. That is, the axial lengths 9, 19 of the fibers 10, 20, respectively, are comparable to the overall conduit length 32a in the water supply system 30. Further, the water sanitizing system 50 includes a UV light source 4 configured to inject UV light rays 1 into the first end 10a, 20a of fibers 10, 20 via the delivery fiber 5. The delivery fiber 5 can be routed through a port in the conduit 32 for this purpose. These UV light rays 1 then propagate along the fibers 10, 20 in the direction of the second ends 10b, 20b of these fibers.

In some embodiments, water sanitizing system 50 can be employed to sanitize water in the conduit 32 by killing or otherwise inhibiting the growth of bacterial organisms 42 in the water and/or the conduit 32. UV light rays 1 are directed from the UV light source 4 into the delivery fiber 5 and then into the first ends 10a, 20a of light-diffusing optical fibers 10, 20 located within the water supply system 30. As depicted in FIG. 3, these UV light rays 1 are then scattered in substantially radial directions at the plurality of scattering sites 3 (see FIGS. 1, 1A, 2 and 2A) out of the fibers 10 and 20 and into the water within the conduit 32. These scattered UV light rays 7 then interact with the bacterial organisms 42, killing them or otherwise inhibiting their growth, and thereby sanitizing the water within the conduit 32. A primary advantage of the water sanitizing system 50 is that it can provide its water sanitizing function along the entire conduit length 32a, as the scattered UV light rays 7 propagate throughout the overall length 9, 19 of the light-diffusing optical fibers 10, 20 toward the second ends 10b, 20b, respectively. As such, bacterial organisms 42 located in different sections of conduit 32 can be killed or otherwise prevented from further growth.

In other embodiments, as shown in FIG. 3, water sanitizing system 50 can be employed to purify water in the conduit 32 by breaking down pesticides 44 in the water. UV light rays 1 are directed from the UV light source 4 into the delivery fiber 5 and then into the first end 10a of light-diffusing optical fibers 10 located within the water supply system 30. As depicted in FIG. 3, these UV light rays 1 are then scattered in substantially radial directions at the plurality of scattering sites 3 (see FIGS. 1 and 1A) out of the fibers 10, through the photocatalyst region 6a, and into the water in the conduit 32. These scattered UV light rays 7 then interact with the photocatalyst region 6a and pesticides 44 to break the pesticides 44 down through photocatalytic reactions, thereby purifying the water within the conduit 32. Here, an advantage of water sanitizing system 50 is that it can provide its water purifying function along the entire conduit length 32a, as the scattered UV light rays 7 propagate throughout the overall length 9 (in the direction of the second end 10b) of the light-diffusing optical fibers 10. Accordingly, pesticides 44 located in the water at high concentration levels in multiple sections of the conduit 32 can be broken down.

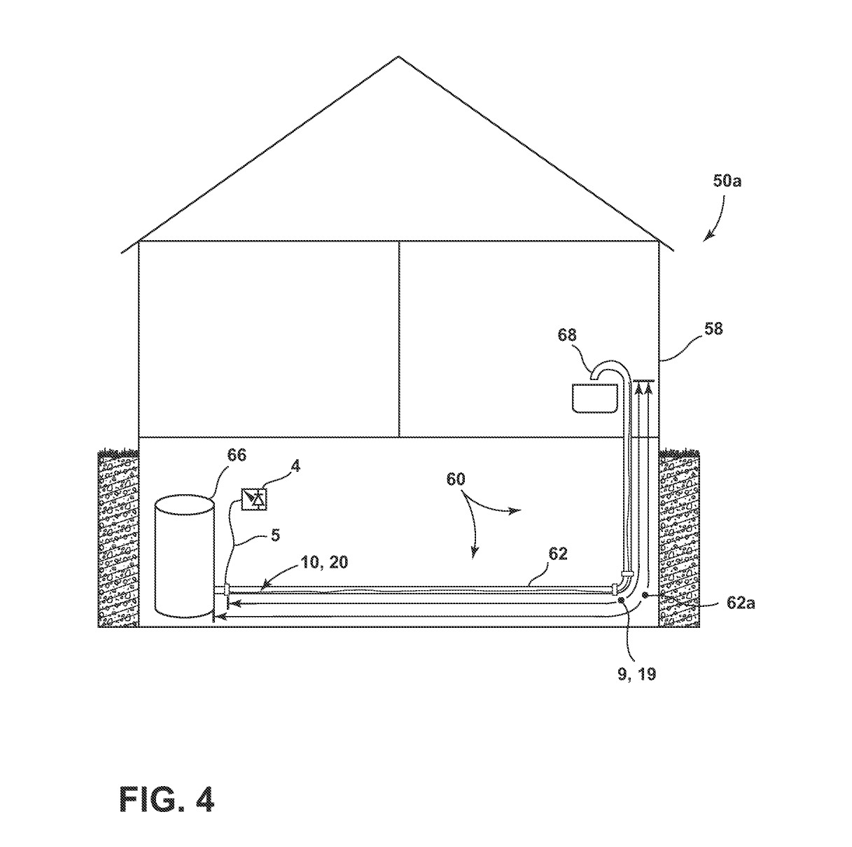

In another exemplary embodiment, water sanitizing system 50a is depicted in FIG. 4. Sanitizing system 50a utilizes light-diffusing optical fibers 10, 20 (see FIGS. 1, 1A, 2, 2A). The sanitizing system 50a can be employed within a plumbing system 60 located in a residence 58. The plumbing system 60 includes a water supply 66 and a water outlet 68, connected via a conduit 62 having a conduit length 62a. The optical fibers 10, 20 are routed within the conduit 62 such that their axial length 9, 19 substantially spans the conduit length 62a. Further, a UV light source 4 is connected to the optical fibers 10, 20 via a delivery fiber 5. The operation of water sanitizing system 50a shown in FIG. 4 is consistent with the sanitizing system 50 described earlier.

As such, water sanitizing system 50a can be employed to sanitize and purify water contained throughout the conduit length 62a of the conduit 62 residing within the plumbing system 60. According to some embodiments, the light-diffusing optical fibers 10, 20 employed in sanitizing system 50a can be tailored to provide further attenuation-induced scattering at locations of interest within the plumbing system 60. For example, the scattering sites 3 can be concentrated with the regions of the fibers 10, 20 in proximity to the water supply 66, thereby increasing the quantity of scattered UV light rays 7, and overall UV light propagation into the water at this location.

In some other embodiments, the water sanitizing system 50a can be employed within the plumbing system 60 well after the construction of residence 58. Light-diffusing optical fibers 10, 20 are particularly small in diameter relative to the typical diameter of plumbing components in a residential plumbing system. As such, sanitizing system 50a can be easily routed and installed within a residence 58. In some embodiments, parachute-like devices can be temporarily installed at the second ends 10b, 20b of the fibers and used to deploy the fibers 10, 20 within the conduit 62 of the plumbing system 60 (not shown). Air is directed against the parachute-like device to move the fibers 10, 20 within the conduit 62. Once the desired location of the fibers 10, 20 is obtained, the parachute-like devices are then removed.

Further, the relatively low profile of fibers 10, 20 employed in the sanitizing system 50a will not substantially affect the overall water flow characteristics in the plumbing system 60. In addition, the UV light source 4 employed with water sanitizing system 50a uses very little energy with virtually no noise emission.

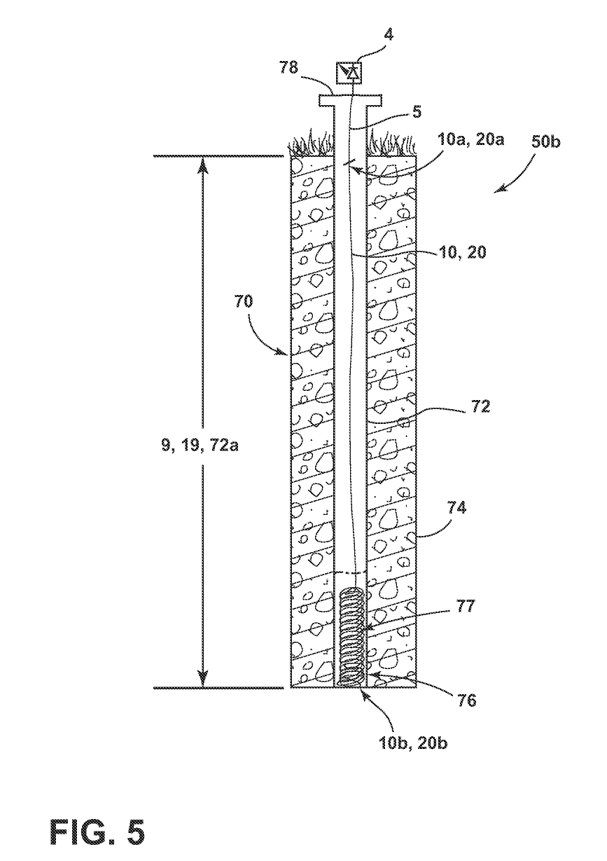

In a further exemplary embodiment, a water sanitizing system 50b is depicted in FIG. 5. Here, the sanitizing system 50b can be employed within a well system 70. The well system 70 includes a well 74, bottommost portion 76 of the well, and a well outlet 78. A conduit 72 connects the bottommost portion 76 of the well 74 to the outlet 78. Further, the conduit 72 is configured to deliver water from the bottommost portion 76 of the well 74 to the outlet 78. As understood by those with ordinary skill in the field, other equipment not shown in FIG. 5 can be employed to draw water from the bottommost portion 76 of the well 74 toward the outlet 78.

As shown in FIG. 5, the light-diffusing optical fibers 10, 20 employed in water sanitizing system 50b are deployed within the well system 70 in the conduit 72 such that their axial lengths 9, 19 substantially span the conduit length 72a of the well 74. In particular, the first ends 10a, 20a of the fibers 10, 20, respectively, are located in proximity to the well outlet 78. The second ends 10b, 20b of the fibers are located in the bottommost portion 76 of the well 74. More specifically, the second ends 10b, 20b of the fibers 10, 20 are configured in the form of a nest 77 in the bottommost portion 76 of the well. In some embodiments, the nest 77 can be in the form of a tightly-wound coil. In general, the nest 77 should serve to increase the overall surface area of the ends 10b, 20b of the fibers 10, 20 in contact with water located in the bottommost portion 76 of the well 74.

Further, a UV light source 4 is connected to the optical fibers 10, 20 via a delivery fiber 5. The operation of water sanitizing system 50b shown in FIG. 5 is consistent with the sanitizing system 50 described earlier. As such, system 50b can be employed to sanitize and purify water contained throughout the conduit length 72a of the conduit 72 residing within the well system 70. In many embodiments, the light-diffusing optical fibers 10, 20 employed in sanitizing system 50b are tailored to provide higher attenuation-induced scattering levels in the bottommost portion 76 of the well 74. This is because the bottommost portion 76 of the well 74 typically contains water from the well source, potentially with unacceptable bacteria and/or pesticide concentrations. For example, the fibers 10, 20 can be tailored such that their plurality of scattering sites 3 are concentrated in the nest 77 portions of these fibers at their second ends 10b, 20b. This has the effect of increasing the quantity of scattered UV light rays 7 that scatter into the water at the bottommost portion 76 of the well 74, thereby enhancing the water purification and sanitizing function of the system 50b at this location.

In some embodiments, the water sanitizing system 50b can be installed in a well 74, after the construction of the well system 70. One reason for the relative ease of installation of system 50 is that light-diffusing optical fibers 10, 20 are small in diameter, particularly in view of the relatively large diameter of a well 74. Furthermore, a weight can be attached to the second ends 10b, 20b of the fibers 10, 20 in proximity to the nest 77. The fibers 10, 20 can then be released at the outlet 78 of the well 74, and gravity can act on the weight to move the fibers 10, 20 down through the conduit 72 into a final, desired location. In addition, the relatively small diameter of the fibers 10, 20 will not impede the flow of water through conduit 72 within the well system 70.

It will be apparent to those skilled in the art that various modifications and variations can be made without departing from the spirit or scope of the claims.

What is claimed is:

1. An optical fiber for sanitizing a water supply system, comprising:
a light-diffusing optical fiber that includes:
(a) a length,
(b) a core region comprising fused silica having a plurality of scattering sites, and
(c) a cladding over the core region, the cladding having an outer photocatalyst region doped with a metal oxide,
wherein the fiber is configured to propagate ultraviolet light rays along the length, and scatter the ultraviolet light rays in substantially radial directions out of the core region of the fiber at the plurality of scattering sites, through the photocatalyst region.

2. The optical fiber according to claim 1, wherein the fiber is further configured to scatter the ultraviolet light at an attenuation loss of less than or equal to about 20 dB/m.

3. The optical fiber according to claim 1, wherein the photocatalyst region within the cladding is defined by a thickness ranging from about 0.1 μm to about 10 μm.

4. The optical fiber according to claim 3, wherein the metal oxide photocatalyst is titanium dioxide and the photocatalyst region is doped with the titanium dioxide at a concentration ranging from about 1% to about 20% by weight.

5. The optical fiber according to claim 4, wherein the plurality of scattering sites comprise air pockets at a volume fraction of about 0.1% to about 30%.

6. An optical fiber for sanitizing a water supply system, comprising:
a light-diffusing optical fiber that includes:
(a) a length,
(b) a core region comprising fused silica having a plurality of scattering sites, and
(c) a cladding over the core region that comprises a polymer coating,
wherein the fiber is configured to propagate ultraviolet light rays along the length, and scatter the ultraviolet light rays in substantially radial directions out of the core region of the fiber at the plurality of scattering sites.

7. The optical fiber according to claim 6, wherein the fiber is further configured to scatter the ultraviolet light at an attenuation loss of less than or equal to about 20 dB/m.

8. The optical fiber according to claim 6, wherein the plurality of scattering sites comprises air pockets at a volume fraction of about 0.1% to about 30%.

9. A water sanitizing system, comprising:
a water supply system having a water supply conduit with a conduit length;
a light-diffusing optical fiber in the conduit that substantially spans the conduit length; and
an ultraviolet light source configured to inject ultraviolet light rays into the optical fiber,
wherein the light-diffusing optical fiber includes:
(a) a first end and a second end, the ends defining a fiber length,
(b) a core region comprising fused silica having a plurality of scattering sites, and
(c) a cladding over the core region, the cladding having an outer photocatalyst region doped with a metal oxide,
and further wherein the fiber is configured to propagate the ultraviolet light rays from the first end toward the second end of the fiber, and scatter the ultraviolet light rays in substantially radial directions out of the core region of the fiber at the plurality of scattering sites, and through the photocatalyst region.

10. The water sanitizing system according to claim 9, wherein the optical fiber is configured to treat bacterial organisms and pesticides substantially along the conduit length.

11. The water sanitizing system according to claim 9, wherein the water supply system is a plumbing system having a water supply and an outlet, and the water supply conduit is configured to deliver water from the supply to the outlet.

12. The water sanitizing system according to claim 9, wherein the water supply system is a well system having a well and an outlet, and the water supply conduit is configured to deliver water from the well to the outlet, and further wherein the second end of the fiber is in the form of a nest within a bottom portion of the well.

13. The water sanitizing system according to claim 9, wherein the fiber is further configured to scatter the ultraviolet light at an attenuation loss of less than or equal to about 20 dB/m.

14. The water sanitizing system according to claim 13, wherein the photocatalyst region within the cladding is defined by a thickness ranging from about 0.1 μm to about 10 μm.

15. The water sanitizing system according to claim 14, wherein the metal oxide photocatalyst is titanium dioxide and the photocatalyst region is doped with the titanium dioxide at a concentration ranging from about 1% to about 20% by weight.

16. A water sanitizing system, comprising:
a water supply system having a water supply conduit with a conduit length;
a light-diffusing optical fiber in the conduit that substantially spans the conduit length; and
an ultraviolet light source configured to inject ultraviolet light rays into the optical fiber,
wherein the light-diffusing optical fiber includes:
(a) a first end and a second end, the ends defining a fiber length,
(b) a core region comprising fused silica having a plurality of scattering sites, and
(c) a cladding over the core region that comprises a polymer coating, and
further wherein the fiber is configured to propagate the ultraviolet light rays from the first end toward the second end of the fiber along the fiber length, and scatter the ultraviolet light rays in substantially radial directions out of the core region of the fiber at the plurality of scattering sites.

17. The water sanitizing system according to claim 16, wherein the optical fiber is configured to treat bacterial organisms substantially along the conduit length.

18. The water sanitizing system according to claim 16, wherein the water supply system is a plumbing system having a water supply and an outlet, and the water supply conduit is configured to deliver water from the supply to the outlet.

19. The water sanitizing system according to claim 16, wherein the water supply system is a well system having a well and an outlet, and the water supply conduit is configured to deliver water from the well to the outlet, and further wherein the second end of the fiber is in the form of a nest within a bottom portion of the well.

20. The water sanitizing system according to claim 16, wherein the fiber is further configured to scatter the ultraviolet light at an attenuation loss of less than or equal to about 20 dB/m.
